(19) United States
(12) Patent Application Publication
(10) Pub. No.: US 2002/0183769 A1
(43) Pub. Date: Dec. 5, 2002

(54) Medical grafting methods and apparatus

(75) Inventors: William J. Swanson, St. Paul, MN (US); Rick Cornelius, Wayzata, MN (US); Paul J. Hindrichs, Plymouth, MN (US); Alex A. Peterson, Maple Grove, MN (US)

Correspondence Address: FISH & NEAVE, 1251 AVENUE OF THE AMERICAS, 50TH FLOOR, NEW YORK, NY, 10020-1105, US

(73) Assignee: ST. JUDE MEDICAL ATG, INC.

(21) Appl. No.: 10/158,436

(22) Filed: May 28, 2002

Related U.S. Application Data
(60) Provisional application No. 60/294,459, filed on May 30, 2001.

(51) Int. Cl. A61B 17/08

(52) U.S. Cl. 606/153

(57) ABSTRACT

Methods and apparatus for making an anastomotic connection between a first conduit and a second conduit are provided. A connector is mounted annularly around the distal end portion of an inflatable balloon. Upon inflation of the balloon, the connector enlarges radially and shortens axially such that first and second members of the connector engage the first and second conduits, respectively, and draw the two conduits together to create a seal therebetween. Additional apparatus may be provided to reduce the tension on the first conduit (e.g., a graft conduit) and/or to define a kink-free path for the first conduit to follow after making the anastomotic connection.

This application claims the benefit of U.S. provisional patent application No. 60/294,459, filed May 30, 2001, which is hereby incorporated by reference herein in its entirety.

### BACKGROUND OF THE INVENTION

This invention relates to medical grafting methods and apparatus and, more particularly, to methods and apparatus for making anastomotic connections between tubular body fluid conduits in a patient.

There are many medical procedures in which it is necessary to make an anastomotic connection between two tubular body fluid conduits in a patient. An anastomotic connection (or anastomosis) is a connection which allows body fluid flow between the lumens of the two conduits that are connected, preferably without allowing body fluid to leak out of the conduits at the location of the connection. As just one example of a procedure in which an anastomosis is needed, in order to bypass an obstruction in a patient's coronary artery, a tubular graft supplied with aortic blood may be connected via an anastomosis to the coronary artery downstream from the obstruction. The anastomosis may be between the end of the graft and an aperture in the side wall of the coronary artery (a so-called end-to-side anastomosis), or the anastomosis may be between an aperture in the side wall of the graft and an aperture in the side wall of the coronary artery (a so-called side-to-side anastomosis). The graft may be natural conduit, synthetic conduit, or a combination of natural and synthetic conduits. If natural conduit is used, it may be wholly or partly relocated from elsewhere in the patient (e.g., wholly relocated saphenous vein graft (“SVG”) or partly relocated internal mammary artery (“IMA”)). Alternatively, no relocation of the graft may be needed (e.g., a length of vein on the heart becomes a “graft” around an obstruction in an immediately adjacent coronary artery). More than one anastomosis may be needed. For example, a second anastomosis may be needed between an upstream portion of the graft conduit and the aorta or the coronary artery upstream from the obstruction in that artery. Again, this second anastomosis may be either an end-to-side anastomosis or a side-to-side anastomosis. Alternatively, no second upstream anastomosis may be required at all (e.g., if the graft is an only-partly-relocated IMA).

The current most common technique for making an anastomosis is to manually suture the two tubular body fluid conduits together around an opening between them. Manual suturing is difficult and time-consuming, and the quality of the anastomosis that results is highly dependent on the skill of the person doing the suturing.

Various types of mechanical connectors have been developed to reduce or eliminate the need for suturing, but improvements are constantly sought for such mechanical connectors with respect to considerations such as ease and speed of use, ease of manufacture, strength and permanence of the resulting connection, etc.

Accordingly, it would be desirable to provide methods and apparatus for making anastomotic connections between tubular body fluid conduits in a patient.

### SUMMARY OF THE INVENTION

In accordance with the invention, an apparatus including a connector is provided to create a hollow annular anastomotic connection between tubular body fluid conduits in a patient. A particular application of the invention is to join a graft conduit to a body tissue conduit in a patient in a side-to-side anastomosis. The connector has a first set of members that engage a first conduit (e.g., the graft conduit) and a second set of members that engage a second conduit (e.g., the body tissue conduit).

The connector is mounted on a balloon catheter which, when pressurized, expands to a significant extent at the distal end thereof. The balloon enlarges the connector positioned at the distal end portion of the balloon to create the anastomosis.

The second set of members is covered by a nosecone balloon of a nosecone assembly to prevent trauma to the second conduit when the connector is introduced. The nosecone assembly has a flexible structure which may change configuration to expose the second set of members after insertion into the second conduit and to allow removal of the nosecone assembly after deployment of the connector.

A method for creating the anastomosis may include introducing a hollow annular connector into the first conduit. A first axial portion of the connector may be disposed inside the first conduit, and a second axial portion of the connector may extend out of the graft conduit via an aperture in the first conduit. The aperture in the first conduit may be made in the side wall of the first conduit proximal to the distal end of the conduit.

At the operative site, a second aperture may be made in the side wall of the second conduit. The first and second apertures may be approximated so that the second axial portion of the connector extends into the second conduit via the second aperture.

The connector may be deformed so that it presses together the side walls of the first and second conduits annularly around the first and second apertures. The connector may enlarge radially and shorten axially upon expansion. A selectively inflatable balloon may be used to deform the connector.

A U-shaped channel may be provided to reduce the tension on a graft conduit and to define a kink-free path for the graft conduit to follow. The length (e.g., short or long) and shape (e.g., straight or curved) of the U-shaped channel may be varied depending on the desired result. For example, a long, curved channel may be positioned over top of a graft conduit to define a curved path which may reduce the likelihood that the graft conduit will kink. In another example, a short, straight channel may be positioned over top of a graft conduit to reduce the effect of tension on the graft conduit.

### BRIEF DESCRIPTION OF THE DRAWINGS

FIG. 1 is a planar development of the structure of an illustrative embodiment of a connector constructed in accordance with the invention.

FIG. 2 is a perspective view of the connector which is shown in planar development in FIG. 1 in accordance with the invention.

FIG. 3 is a planar development of the connector of FIG. 1 in another configuration in accordance with the invention.

FIG. 4 is a perspective view similar to FIG. 2 of the connector of FIG. 1 in another configuration in accordance with the invention.

FIG. 5A is a sectional view of the connector of FIG. 1 with additional illustrative apparatus for use in delivering and deploying the connector in accordance with the invention.

FIG. 5B is another sectional view of the apparatus of FIG. 5A in accordance with the invention.

FIG. 6 is a perspective view of additional apparatus in accordance with the invention.

FIG. 7 is a sectional view of the apparatus of FIG. 6, illustrated with a first conduit, in accordance with the invention.

FIG. 8 is a sectional view similar to FIG. 7, illustrated with the apparatus of FIGS. 5A-5B, in accordance with the invention.

FIG. 9 is a sectional view of the apparatus of FIGS. 5A-5B illustrating an early stage of a procedure in accordance with the invention.

FIG. 10 is a view similar to FIG. 9 illustrating a further stage of a procedure in accordance with the invention.

FIG. 11 is a view similar to FIG. 10 illustrating a later stage of a procedure in accordance with the invention.

FIG. 12 is a view similar to FIG. 11 illustrating a still later stage of a procedure in accordance with the invention.

FIG. 13 is a view similar to FIG. 12 illustrating yet another stage of a procedure in accordance with the invention.

FIG. 14 is a view similar to FIG. 5A illustrating additional illustrative apparatus in accordance with the invention.

FIG. 15 is a view similar to FIG. 14 illustrating additional illustrative apparatus in accordance with the invention.

FIG. 16 is a simplified sectional view of illustrative apparatus for use with a graft conduit in a patient in accordance with the invention.

FIG. 17 is a view similar to FIG. 16 of another illustrative embodiment of apparatus for use with a graft conduit in a patient in accordance with the invention.

FIG. 18 is a simplified elevational view of one embodiment of the apparatus of FIGS. 16 and 17 in accordance with the invention.

FIG. 19 is a simplified elevational view of another embodiment of the apparatus of FIGS. 16 and 17 in accordance with the invention.

FIGS. 20-23 are planar developments of other illustrative embodiments of a connector constructed in accordance with the invention.

### DETAILED DESCRIPTION OF THE PREFERRED EMBODIMENTS

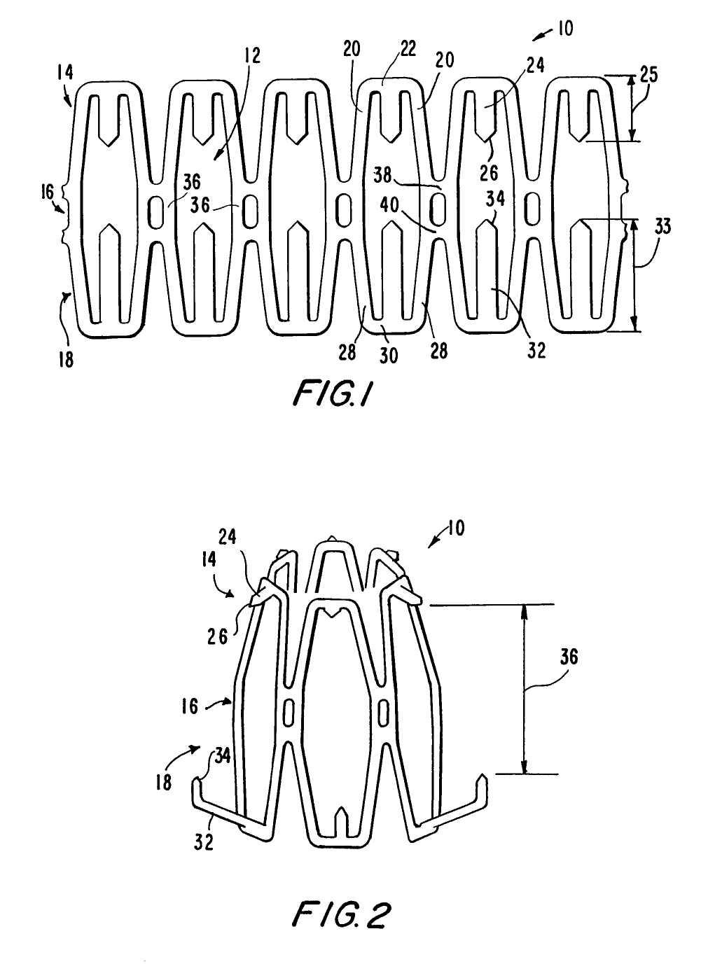

FIG. 1 shows a planar development of what is actually, preferably, an integral, one-piece (unitary), annular connector 10. In particular, the left and right edges of the structure shown in FIG. 1 are actually, preferably, joined to and integral with one another. Thus, the actual structure is as shown in FIG. 2, although FIG. 1 is useful to more clearly reveal certain details of various features of connector 10.

A particularly preferred material for connector 10 is stainless steel (e.g., 316 stainless steel). Other examples of suitable materials include tantalum, tungsten, platinum, other steels, and nickel titanium alloy (“nitinol”). Connector 10 may be advantageously produced by starting with a single, unitary metal tube, such as a hypotube, and removing selected material until only the structure shown in FIG. 2 remains. For example, laser cutting may be used to remove material from the starting tube in order to produce connector 10.

Connector 10 may be described as including annularly spaced cell portions 12. According to one embodiment, connector 10 includes six repeating cell portions 12. The connector may have fewer or more than six of cell portions 12, depending on the diameter of the tube used to manufacture connector 10 and the resulting enlarged diameter desired.

Each cell portion 12 includes a pair of annularly spaced members 20. The distal ends of members 20 are connected to one another at annularly extending member 22. A pair of members 20 and a member 22 define the distal portion 14 of each cell portion 12. Each cell portion 12 also includes a pair of annularly spaced members 28. The proximal ends of members 28 are connected to one another at annularly extending member 30. A pair of members 28 and a member 30 define the proximal portion 18 of each cell portion 12. Each cell portion 12 also includes a pair of annularly spaced members 36. The proximal ends of members 36 are connected to the distal ends of members 28, and the distal ends of members 36 are connected to the proximal ends of members 20. A pair of members 36 defines the medial portion 16 of each cell portion 12.

A cell portion 12 may be connected to an annularly adjacent cell portion 12 by members 38 and 40. Connecting annularly adjacent cell portions 12 at more than one location (e.g., using two members 38 and 40) improves the rigidity of connector 10. For example, with only one connection between annularly adjacent cell portions 12, a cell portion 12 has a tendency to twist with respect to the two annularly adjacent cell portions. However, with two connections between annularly adjacent cell portions 12 (e.g., members 38 and 40), a cell portion 12 is prevented from twisting with respect to the two annularly adjacent cell portions. Although two members 38 are shown in FIG. 1, it will be appreciated that annularly adjacent cell portions 12 may be connected to one another using any number of members 38.

Some or all of the cell portions 12 may include a distal member 24 that in this case has a free end portion 26 that is sharply pointed and that points toward proximal portion 18. Distal member 24 may be connected to annularly extending member 22. A typical distal member 24 may have a length 25 in a range from about 0.010 inches to about 0.020 inches. (It should be noted that length 25 includes the width of member 22.) However, the dimensions of distal member 24 may be altered according to the wall thickness of the conduits to be joined. Each of distal members 24 is deflectable radially outward from the remainder of the structure of connector 10, as shown, for example, in FIG. 2.

Some or all of the cell portions 12 may also include a proximal member 32 that in this case has a free end portion 34 that is sharply pointed and that points toward distal portion 14. Proximal member 32 may be connected to annularly extending member 30. A typical proximal member 32 may have a length 33 in a range from about 0.020 inches to about 0.040 inches. (It should be noted that length 33 includes the width of member 30.) However, the dimensions of proximal member 32 may be altered according to the thickness of the conduits to be joined. Each of members 32 is deflectable radially outward from the remainder of the structure of connector 10, as shown, for example, in FIG. 2.

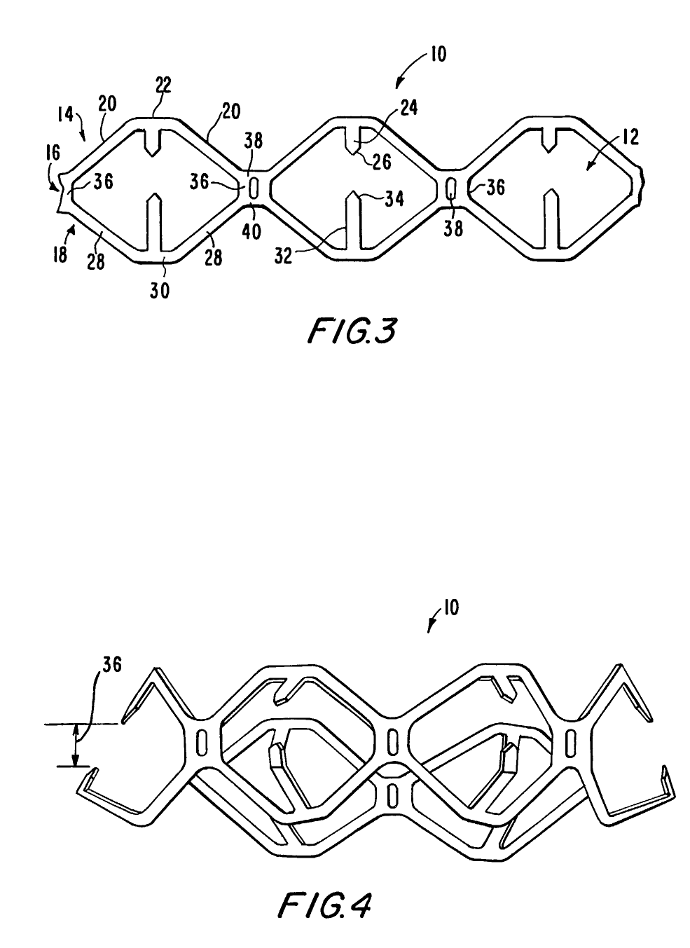

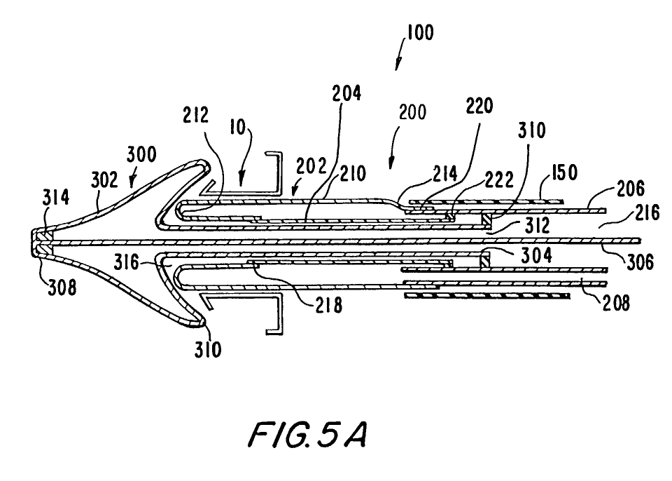

The above-mentioned outward deflection of distal members 24 and proximal members 32 may be produced by putting connector 10 on a mandrel and prying members 24 and 32 radially outward. As shown in FIG. 2, in addition to the radially outward deflection of proximal members 32, a portion of each proximal member 32 that includes free end portion 34 may be bent such that free end portion 34 points toward distal portion 14 of connector 10. Following deflection of members 24 and 32, an initial axial spacing 36 may be defined therebetween. Spacing 36 may vary depending on the wall thicknesses of the two conduits to be joined by connector 10. Also following deflection of members 24 and 32, an initial distal member diameter and an initial proximal member diameter may be defined by the tips of the free end portions of distal members 24 and proximal members 32, respectively. These diameters are “initial” diameters in contrast to the “expanded” diameters defined by the tips of members 24 and 32 following expansion of connector 10 (see, for example, FIG. 4). The initial distal member diameter, for example, may be defined by the pointed tips of free end portions 26. The initial proximal member diameter, for example, may be defined by the pointed tips of free end portions 34.

The diameters defined by distal members 24 and proximal members 32 of connector 10 are one aspect of the invention that allows the members to engage and penetrate the two conduits to be connected upon expansion of the connector, rather than manually piercing a first conduit (e.g., graft conduit) onto the proximal members of the connector prior to expanding the connector within a second conduit (e.g., a coronary artery). This aspect of the invention will be described in greater detail herein.

Connector 10 is preferably annealed. Connector 10 may also be used in the full hard or partially hard state. Connector 10 also typically requires other processing appropriate for an implantable device such as, for example, polishing, passivation, cleaning, and sterilizing.

FIGS. 3 and 4 illustrate the enlarged condition of connector 10. Connector 10 is formed in such a way that it is annularly enlargeable (e.g., by inflation of a balloon that is temporarily disposed inside the connector structure, as will be described in greater detail herein).

A planar development of the annularly enlarged condition of connector 10 is shown in FIG. 3. The annular enlargeability of connector 10 is provided by annularly expanding cell portions, such as cell portions 12 described above. In this way, connector 10 is annularly enlargeable by annularly enlarging some or all of cell portions 12.

It will be appreciated that as connector 10 annularly enlarges, it generally axially shortens. In other words, as cell portions 12 widen in the annular direction, the cell portions shorten in the axial direction. As connector 10 is enlarged in position to join two conduits together, it is desirable for the distal portion 14 and the proximal portion 18 to deflect radially outward to a greater diameter than the diameter associated with medial portion 16. The overall annular enlargement of connector 10 along with the relatively greater enlargement of distal portion 14 and proximal portion 18 together decrease the axial spacing between distal members 24 and proximal members 32, resulting in the reduced axial spacing 36 shown in FIG. 4.

Upon annular enlargement of connector 10, distal members 24 and proximal members 32 may engage and penetrate the inner surfaces of the two conduits to be connected and press the side walls of the two conduits together, creating a seal therebetween. The side walls of the two conduits (e.g., a graft conduit and a body tissue conduit) may be pressed together annularly around the apertures in the side walls of each of the two conduits. Axial spacing 36 after annular enlargement of connector 10 may be slightly less than the sum of the wall thicknesses of the two conduits held together by members 24 and 32. For example, members 24 and 32 may penetrate the walls of the conduits. Also, members 24 and 32 may hold the side walls of the conduits together to form a seal therebetween such that the side walls of the two conduits are slightly compressed at the location of the anastomosis.

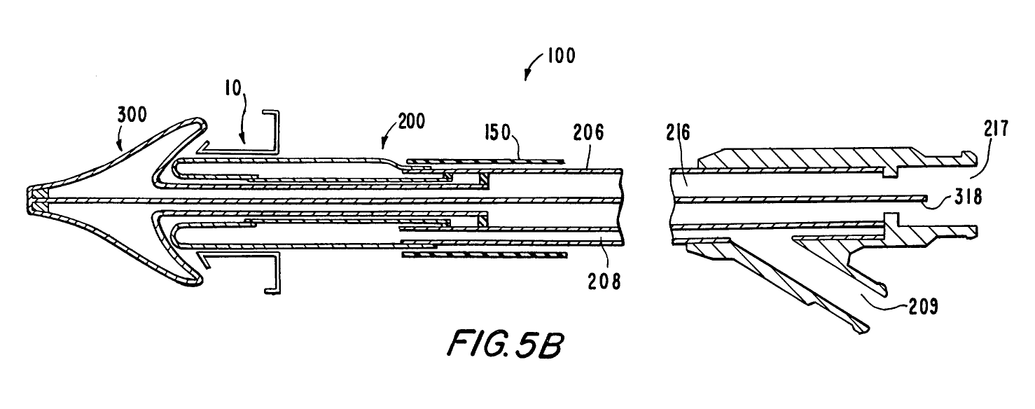

An illustrative apparatus 100 for delivering connector 10 and a first conduit to an operative site and for making a hollow annular anastomotic connection between the first conduit and a second conduit is shown in FIGS. 5A and 5B. The first conduit is typically a graft conduit and may be a natural conduit, such as a saphenous vein graft (“SVG”), a synthetic conduit, or a combination thereof. The second conduit is typically a patient's natural body tissue conduit, such as a coronary artery. FIG. 5A is a sectional view of the distal portion of apparatus 100, and FIG. 5B is a sectional view of the entire apparatus 100.

As shown in FIG. 5A, apparatus 100 may include a number of component elements for delivery and deployment of a connector and a first conduit to the site of an anastomosis. One of the component elements of apparatus 100 is balloon catheter 200, which is useful for enlarging connector 10 to join the first and second conduits (e.g., a graft conduit and a body tissue conduit) at the site of the anastomosis. Another component element of apparatus 100 is nosecone assembly 300, which is useful to assist in insertion of apparatus 100 into an aperture in the second conduit and to shield connector 10 during such insertion to avoid damaging the second conduit.

As mentioned above, balloon catheter 200 is useful for enlarging connector 10 to join first and second conduits at the site of the anastomosis. Balloon catheter 200 may include balloon 202, balloon shaft 204, and catheter 206. Catheter 206 may be a dual-lumen catheter (i.e., having lumens 208 and 216) constructed of a biocompatible polymer such as, for example, nylon, polybutylene terephthalate (“PBT”), polyamide copolymers, or any other suitable material. As shown in FIG. 5B, at the extreme proximal end of catheter 206, there may be a port 209 (FIG. 5B) that allows access to lumen 208 of catheter 206. Lumen 208 is in communication with the interior of balloon 202 and introduces fluid to inflate the balloon. At the extreme proximal end of catheter 206, there may also be a port 217 that allows access to lumen 216 of catheter 206. Lumen 216 is in communication with the interior of nosecone balloon 302 and introduces fluid to inflate the nosecone balloon (as will be described in greater detail herein).

Referring back to FIG. 5A, balloon 202 may include a substantially constant diameter portion 210, a distal portion 212, and a proximal portion 214. Balloon 202 may be attached to balloon shaft 204 at balloon bond 218 in an “inverted manner” (e.g., as described in published Patent Cooperation Treaty (“PCT”) patent publication No. WO 01/39672, published Jun. 7, 2001, which is hereby incorporated by reference herein in its entirety). The term “inverted,” as used herein, shall refer to the condition of balloon 202 wherein an inflated portion of the balloon extends beyond balloon bond 218. Thus, distal portion 212 of balloon 202 does not taper gradually as does proximal portion 214, but rather may “double-back” on itself. The inverted attachment configuration of balloon 202 permits connector 10 to be positioned close to distal portion 212 of balloon 202, and still be sufficiently enlarged by balloon 202 when the balloon is expanded to deploy connector 10 (as will be described in greater detail herein).

As described above, balloon 202 is attached to balloon shaft 204 at balloon bond 218. Balloon shaft 204 may be, for example, a metal hypotube (e.g., steel). Balloon bond 218 between balloon 202 and balloon shaft 204 may be, for example, a thermal weld, an adhesive bond, or any other suitable attachment between balloon 202 and balloon shaft 204. Balloon shaft 204 may maintain balloon 202 in a straight configuration. For example, without a rigid shaft such as balloon shaft 204, balloon 202 may be prone to lose the straight shape shown in FIG. 5A when inflated.

Proximal end 214 of balloon 202 is attached to catheter 206 at balloon bond 220. Balloon bond 220 may be, for example, a thermal weld, an adhesive bond, or any other suitable attachment between balloon 202 and catheter 206. In the case of a thermal weld between balloon 202 and catheter 206, a seal may also be created between balloon shaft 204 and catheter 206. For example, some of the material of which catheter 206 is made may melt during the thermal welding process, resulting in a partial seal between catheter 206 and balloon shaft 204.

Balloon shaft 204 and catheter 206 are also attached together at bond 222. Bond 222 may be formed, for example, by boring a hole through catheter 206 at the location of bond 222 and by wicking adhesive through the hole in catheter 206. As described above, balloon shaft 204 and catheter 206 may also be partially bonded together due to bond 220 between balloon 202 and catheter 206.

There are several design consideration with regard to the construction of balloon catheter 200. First, the configuration of balloon 202 should allow the entire apparatus 100 to “wrap down” to a diameter as small as the initial (i.e., unexpanded) inner diameter of connector 10. Second, the configuration of balloon 202 should allow distal end 14 of connector 10 to be positioned at a reduced distance from distal end 212 of the balloon. Third, the configuration of balloon 202 should allow the balloon to expand to a diameter that is large enough to expand connector 10 to the target expanded diameter.

To address the above design considerations, balloon 202 may be manufactured from a material or materials that yield a high-strength, thin-walled balloon. Balloon 202 should be able to withstand a balloon inflation pressure that is sufficient to properly enlarge connector 10. For example, for a 3.0 mm diameter balloon 202, which may be used to create an anastomosis between first and second conduits both having an inner diameter of about 2.0 mm, the balloon should be able to withstand a balloon inflation pressure in a range of about 10 atm to about 25 atm. The balloon inflation pressure used to create a particular anastomosis is dependent on, for example, the geometry of the connector, the desired increase in diameter of the connector, the desired increase in diameter of one or both of the first and second conduits, etc. Another factor is the ability of balloon 202 to produce a predictable diameter when inflated to high pressures. Materials that are suitable for manufacturing a high-strength, thin-walled balloon may include, for example, nylon, polyethylene terephthalate (“PET”), polyamide copolymers, polyimide, PBT, any other suitable material, or a combination of such materials.

To further address the above design considerations, a significant portion of balloon 202 may be mounted in an inverted manner. As described above, distal end 212 of balloon 202 is mounted in an inverted manner with respect to balloon shaft 204. The inverted mounting of balloon 202 results in a large inflated balloon diameter at distal end 212 of the balloon. This design feature is especially important with regard to second conduits having small inner diameters (e.g., of about 2.0 mm or less) to prevent distal end 212 of balloon 202 from dilating the second conduit when it is inflated.

As shown in the FIG., a significant portion of balloon 202 is found distal of balloon bond 218. More particularly, the length of balloon 202 that is inverted may be greater than the length of connector 10. Thus, connector 10 for the most part may be mounted over balloon 202 and nosecone shaft 304 when apparatus 100 is in its wrapped down configuration, rather than mounted over balloon 202, nosecone shaft 304, and additionally balloon shaft 204. This allows apparatus 100 to achieve a smaller profile when it is in the wrapped down configuration. For example, a typical connector 10 may have an initial (i.e., unexpanded) inner diameter in the range from about 0.030 inches to about 0.040 inches. Thus, apparatus 100 must wrap down to an outer diameter that is sufficient to fit annularly within connector 10 (i.e., an outer diameter that is less than or equal to the initial inner diameter of the connector).

Another design consideration with regard to balloon catheter 200 is preventing balloon 202 from dilating the first conduit (e.g., graft conduit) when it is inflated. This is particularly important when the first conduit is a vessel having a small inner diameter (e.g., of about 2.0 mm or less). In such cases, a sleeve may be placed around balloon 202 proximal to connector 10 to avoid dilating the first conduit (see, for example, FIGS. 14-15).

Connector 10 may be placed annularly about balloon 202 of balloon catheter 200. Connector 10 is typically installed when balloon 202 is disposed in its unexpanded configuration. The unexpanded configuration of balloon 202 may define a plurality of folded portions that are expanded upon introduction of fluid into balloon 202 (e.g., from port 209 as shown in FIG. 5B). This may also be referred to herein as a wrapped or wrapped down configuration, as balloon 202 is wrapped around nosecone shaft 304 and balloon shaft 204 for installation.

Connector 10 is oriented such that distal portion 14 (FIG. 1) of the connector is positioned adjacent to distal portion 212 of balloon 202. Certain features may be useful to hold connector 10 in place on balloon 202 (e.g., as described in above-mentioned patent publication No. WO 01/39672). In particular, when connector 10 is mounted adjacent to distal portion 212 of balloon 202, it is important to prevent the connector from slipping forward, where it may not be enlarged as fully as desired because it is positioned over a smaller diameter region of balloon 202. In one embodiment, connector 10 is mounted over balloon 202, which is “pre-inflated,” or inflated to a low pressure to hold the connector in place without enlarging the connector. For example, balloon 202 may be inflated to a pressure of about 3 atm for delivery of connector 10. According to another embodiment, a larger diameter may be heat set in balloon 202 just distal of distal portion 14 (FIG. 1) of connector 10 to prevent the connector from slipping forward. According to yet another embodiment, balloon 202 may be covered with a material having a high coefficient of friction to create higher frictional forces between the balloon and connector 10. A material such as, for example, urethane in the 30D to 60D durometer range may be useful for this purpose. This material may be provided with a separate sleeve or with a co-extrusion of the softer material and the base balloon material at the time of extruding the balloon blank. According to yet another embodiment, nosecone balloon 302 (as will be described in greater detail herein) may be positioned distal of connector 10 to hold the connector in position on balloon 202 at least until the nosecone is deployed (e.g., by introducing fluid into nosecone balloon 302).

Referring now to another component element of apparatus 100, nosecone assembly 300 is useful to assist in insertion of apparatus 100 into an aperture in the second conduit (e.g., a patient's body tissue conduit) and to shield connector 10 during such insertion to avoid damaging the second conduit. Nosecone assembly 300 may include a nosecone balloon 302, a nosecone shaft 304, and an indicator wire 306. Nosecone balloon 302 may be fabricated of a number of materials such as, for example, polyethylene, polyolefin copolymers, ethylene vinyl acetate, urethane, any other suitable material, or a combination of such materials. Nosecone balloon 302 may include a distal tapered portion 308 and a proximal portion 310.

Preferably, nosecone shaft 304 is an extension of nosecone balloon 302 and is made of the same material or materials as nosecone balloon 302 (as listed hereinabove). Thus, nosecone balloon 302 is molded such that nosecone shaft 304 extends proximally from the nosecone balloon. This design feature of nosecone assembly 300 eliminates the need for a separate nosecone shaft that is bonded to the nosecone balloon at, for example, a lap joint between the nosecone balloon and shaft. The flexible nosecone shaft 304, made of flexible nosecone balloon material, results in a reduction of the wrapped down profile of apparatus 100 in comparison to a nosecone assembly having a separate, rigid nosecone shaft structure bonded to the nosecone balloon.

Nosecone shaft 304 is bonded to catheter shaft 206 at nosecone shaft bond 310. Bond 310 may be formed by, for example, boring a hole through catheter 206 at the location of bond 310 and wicking adhesive through the hole. The internal cavity of nosecone balloon 302, nosecone shaft lumen 312, and catheter lumen 216 are preferably in fluid communication with one another. For example, fluid may be introduced into catheter lumen 216 through port 217 (as shown in FIG. 5B) at the extreme proximal end of apparatus 100 and may flow into the internal cavity of nosecone balloon 302 via nosecone shaft lumen 312.

Indicator wire 306 is attached to distal tip 308 of nosecone balloon 302 and extends proximally through nosecone shaft lumen 312 and catheter lumen 216. Wire 306 may be set in place using an adhesive 314. The entire nosecone assembly 300 is flexible and capable of bending to an angle of about 100 degrees or more with respect to the longitudinal axis thereof (see, for example, FIG. 11).

In FIG. 5A, nosecone balloon 302 is illustrated in its “introduction configuration,” or folded configuration. Nosecone balloon 302 also may achieve a “removal configuration,” or unfolded configuration (see, for example, FIG. 12). In its introduction configuration, proximal portion 310 of balloon 302 is folded back in a concave manner and defines an annular recess 316 for receiving connector 10, balloon 202, or the like, as will be described in greater detail herein. The expansion of nosecone balloon 302 is typically achieved by introducing fluid into nosecone balloon 302 via lumens 216 and 312, thereby changing the configuration of nosecone balloon 302 from the introduction configuration to the removal configuration (i.e., from the folded configuration of proximal portion 310 depicted in FIG. 5A to the unfolded condition of proximal portion 310 depicted in FIG. 12). When nosecone balloon 302 moves from the introduction configuration to the removal configuration, the nosecone balloon defines a smaller outer dimension and smooth proximal surface to facilitate removal of the nosecone balloon from the second conduit and connector 10, as will be described in greater detail herein.

Indicator wire 306 moves within nosecone shaft lumen 312 and catheter lumen 216 with distal tip portion 308. Consequently, a proximal length of indicator wire 306 may extend out of catheter 206 at the extreme proximal end of apparatus 100 when nosecone balloon 302 is folded (see, for example, proximal length 318 as shown in FIGS. 5B and 9). When nosecone balloon 302 is unfolded, distal tip portion 308 advances distally with respect to catheter 206. When the distal tip portion 308 advances distally, proximal length 318 of wire 306 is drawn into catheter shaft 206. During the distal advancement of distal tapered portion 308, catheter 206 and nosecone shaft 304 remain stationary. In another embodiment, nosecone balloon 302 could be advanced mechanically, for example, by advancing a substantially rigid indicator wire. In yet another embodiment, nosecone assembly 300 may be manufactured without an indicator wire.

The dimensions of nosecone balloon 302 (i.e., the diameter and length) are selected in order to cover the distal members 24 (FIG. 1) of connector 10 during introduction of apparatus 100 into the second conduit. The diameter of nosecone balloon 302 may be defined by the maximum diameter of nosecone balloon 302 in its folded configuration. Nosecone balloon 302 must be of a diameter sufficient to pass through an aperture in the side wall of the second conduit. The size of the aperture in the side wall of the second conduit may depend on the elasticity of the conduit wall. For example, a relatively elastic conduit may have an aperture with a diameter smaller than the diameter of nosecone balloon 302, while a less elastic conduit may have an aperture with a diameter sized closer to the diameter of the nosecone balloon. Nosecone balloon 302 may pass through the aperture in the side wall of the second conduit, and the aperture may reside annularly around medial portion 16 (FIG. 1) of connector 10 after nosecone balloon 302 has passed entirely through the aperture. Although nosecone balloon 302 may be constructed in various sizes for various uses, a typical nosecone has a diameter that is slightly larger than the initial distal member diameter (i.e., the diameter defined by the tips of free end portions 26 of distal members 24) of connector 10.

The diameter of nosecone balloon 302 is also sized for proper loading of a first conduit onto connector 10. The first conduit may be loaded onto connector 10 such that an aperture in the side wall of the first conduit (i.e., the aperture to be connected to the aperture in the second conduit) resides annularly around medial portion 16 (FIG. 1) of connector 10 (as will be described in more detail herein). Nosecone balloon 302 (i.e., the diameter of nosecone balloon 302) may prevent the aperture from advancing distally, and proximal members 32 (i.e., the diameter defined by proximal members 32) may prevent the aperture from advancing proximally.

While filled with expansion fluid in the unfolded condition (see, for example, FIG. 10), nosecone balloon 302 may define a degree of rigidity. Typically, the rigidity is proportional to the pressure of the expansion fluid. For example, nosecone balloon 302 becomes more flexible as more fluid is drained from the nosecone balloon (e.g., prior to reorienting connector 10 with respect to the second conduit, as shown in FIG. 11).

Nosecone shaft 304 is configured to be axially received in the lumen of the balloon catheter shaft 204. In the folded condition, nosecone balloon 302 is folded about expansion balloon 202 and connector 10. Distal members 24 (FIG. 1) of connector 10 are covered so that the periphery of the aperture in the second conduit does not snag on these members as the connector is inserted into this aperture.

According to another embodiment of the invention, nosecone balloon 302 may be substituted by a solid cap, which covers distal members 24 (FIG. 1) of connector 10 during insertion into the aperture in the second conduit. Additional details of nosecone structures are described in, for example, Swanson et al. U.S. Pat. No. 6,113,612, which is hereby incorporated by reference in its entirety herein.

Apparatus 100 may be used along with a grip 150. Grip 150 may not be a component element of apparatus 100. Rather, grip 150 may be used in conjunction with apparatus 100 by a physician to hold, or “grip,” apparatus 100 during, for example, the loading of a first conduit onto connector 10, delivery of the apparatus and first conduit to an anastomosis site, and deployment of the connector and first conduit at the anastomosis site. Grip 150 may be a tubular structure made of a biocompatible rigid material such as, for example, plastic (e.g., polyethylene, PTFE, etc.), metal (e.g., stainless steel), or any other suitable material.

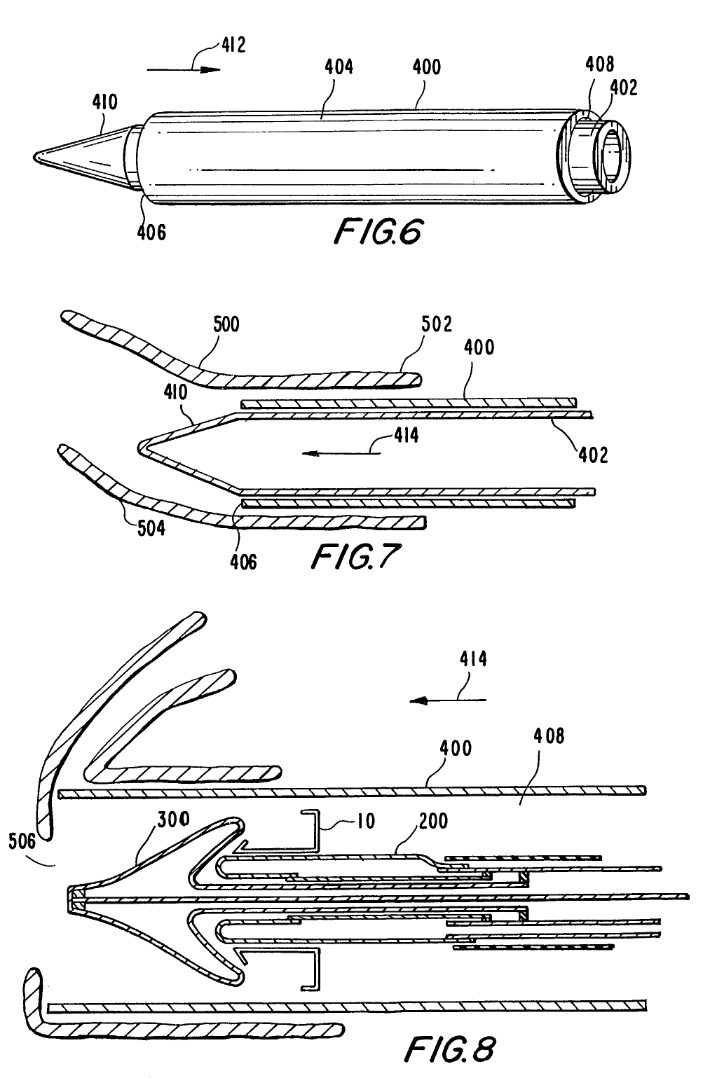

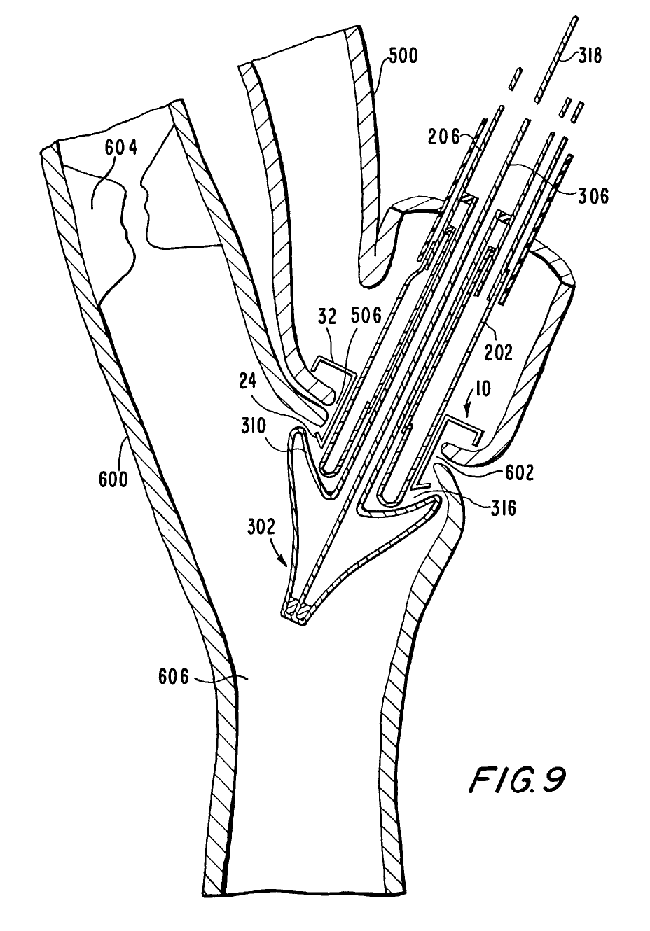

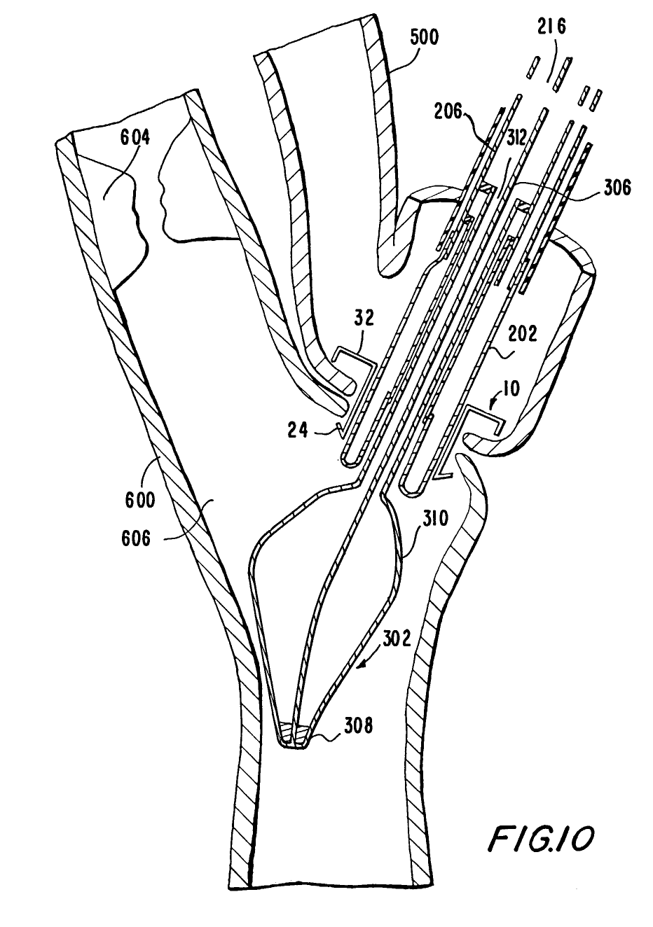

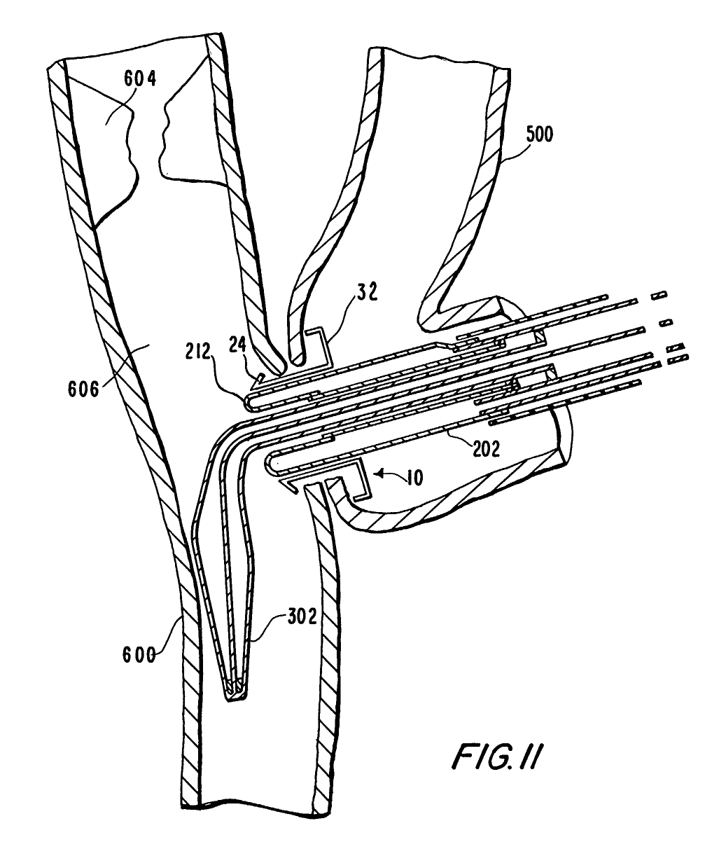

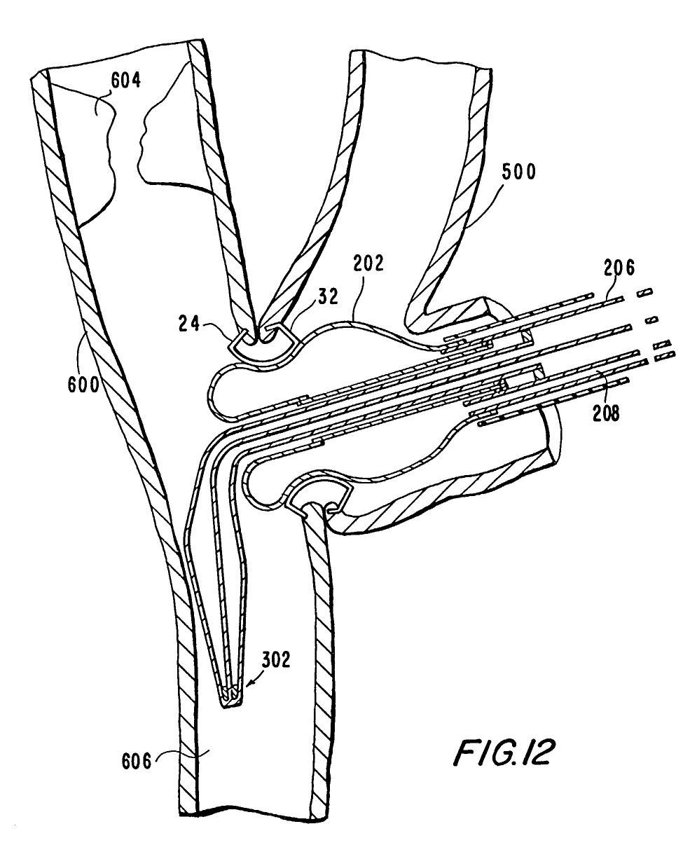

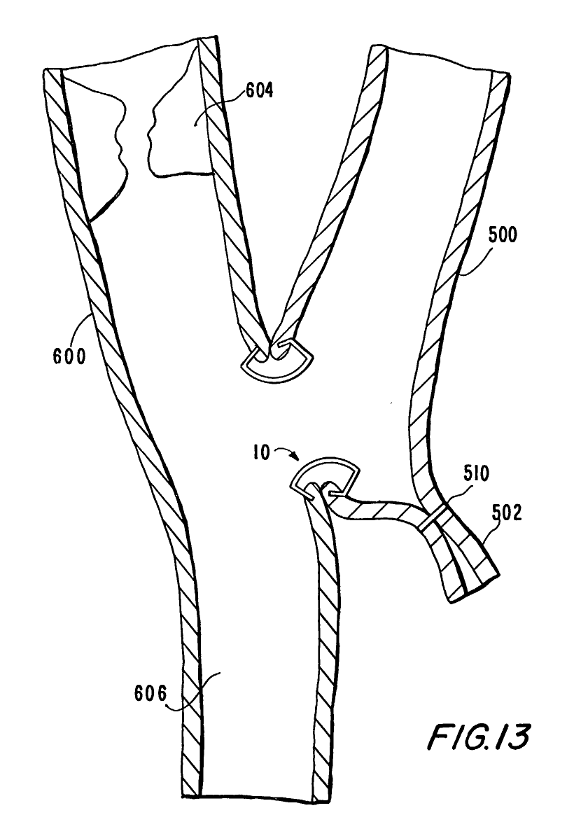

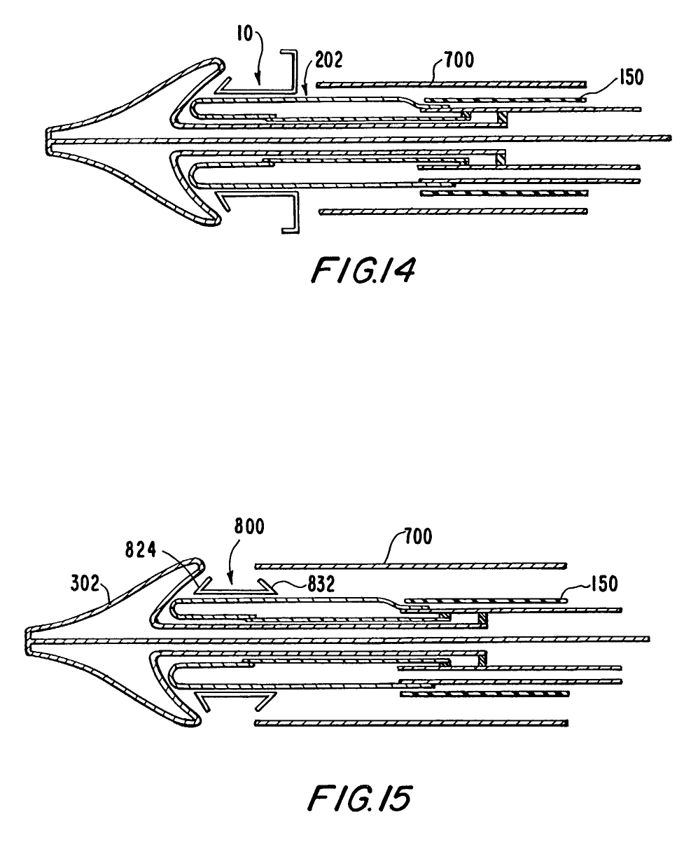

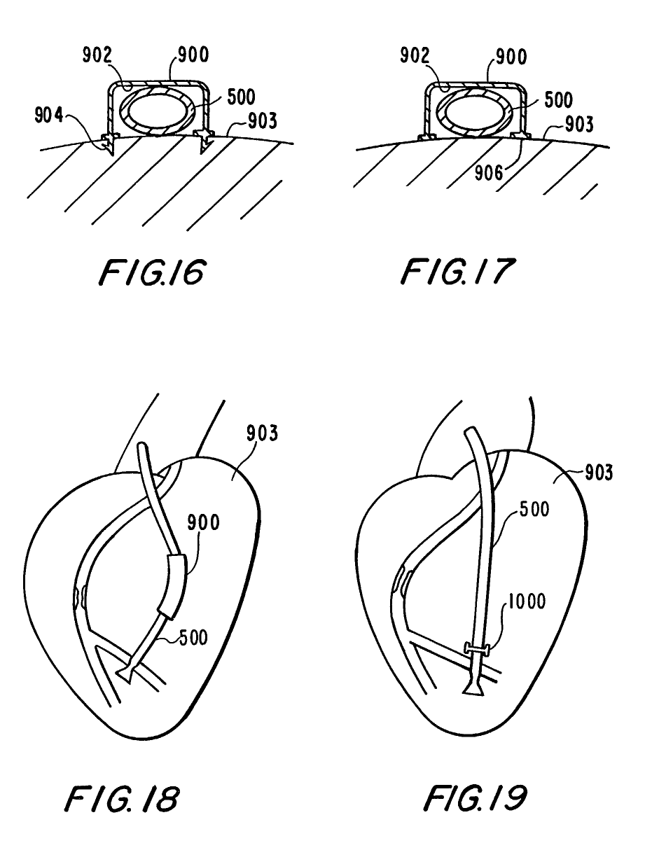

A first conduit may be loaded onto connector 10 prior to deployment of the connector at the anastomosis site. FIG. 6 shows a transfer sheath 400 and rod 402 that may assist in the mounting of the first conduit (e.g., first conduit 500 of FIG. 7) without compromising the delicate intima of the first conduit. Transfer sheath 400 and rod 402 may be fabricated from a low friction, biocompatible polymer such as, for example, polyethylene, PET, or another similar material. Transfer sheath 400 may alternatively be made of metal such as, for example, stainless steel. Rod 402 may be rigid or expandable, as described below. Transfer sheath 400 may have an elongated body portion 404 with a distal end portion 406 and an internal lumen 408. Rod 402, having a tapered end portion 410, is sized to be coaxially positioned within lumen 408 such that tapered end portion 410 extends beyond distal end portion 406 of transfer sheath 400. Tapered end portion 410 may be rigid or may be configured to expand and contract. For example, tapered end portion 410 may be configured to expand to a dimension as large as the outer diameter of transfer sheath 400 for a smooth transition from tapered end portion 410 to body portion 404, and may then be configured to collapse to a smaller dimension to be retracted through internal lumen 408. This allows the first conduit (e.g., first conduit 500 of FIG. 7) to be loaded over transfer sheath 400, in the direction indicated by arrow 412.

Transfer sheath 400 assists a physician by serving as a sizing instrument. The outer diameter of body portion 404 is selected to accommodate the first conduit, such as a graft conduit, having a diameter which is compatible with connector 10. For example, a first conduit that is too narrow will not be able to receive transfer sheath 400 therethrough. Moreover, the internal diameter of the first conduit should be sufficiently large to allow for expansion of balloon 202 (FIG. 5A) and connector 10 (FIG. 1) without dilating the first conduit during such expansion. Alternatively, dilation of the first conduit may be prevented by positioning a sleeve annularly around balloon 202 proximal to connector 10 (as will be described in greater detail herein).

As illustrated in FIG. 7, a first conduit 500 may be positioned over transfer sheath 400. Distal end portion 502 of first conduit 500 is positioned over the transfer sheath 400. Entry through distal end portion 502 allows the remainder of first conduit 500 to be free, which is useful, for example, when the proximal end of first conduit 500 is to be attached to another vessel, such as the aorta of the patient. As illustrated in the FIG., tapered end portion 410 of rod 402 extends distally from transfer sheath 400 to provide a smooth transition as transfer sheath 400 and rod 402 are advanced within the lumen of first conduit 500 in the direction indicated by arrow 414.

When transfer sheath 400 is positioned at location 504 where aperture 506 (as shown in FIG. 8) is to be made in the side wall of first conduit 500, rod 402 is withdrawn proximally, while the transfer sheath remains in position. The wall of first conduit 500 is held taut over distal end 406 of transfer sheath 400. Aperture 506 (FIG. 8) is then made in the side wall of first conduit 500.

Aperture 506 in first conduit 500 may be made by cutting, mechanical dilation, or a combination of both. In one example, an initial aperture is made by cutting first conduit 500 with a blade and then dilating the aperture using a dilator. For example, a 0.045 inch blade may be used, followed by a 0.060 inch dilator, to prepare aperture 506 for a nosecone balloon 302 (FIG. 5A) having a diameter of about 0.063 inches. However, the size of the initial cut and the dilator may be selected based upon the elastic characteristics of first conduit 500 or on any other factor related to the size of the aperture in the first conduit. Other examples of methods and apparatus for creating an aperture in a side wall of a conduit are described in, for example, above-mentioned patent publication No. WO 01/39672. Such methods and apparatus may also be used to create an aperture in a second conduit (e.g., a patient's body tissue conduit).

As illustrated in FIG. 8, balloon catheter 200 and nosecone assembly 300 (i.e., apparatus 100) and connector 10 are advanced in the direction of arrow 414 through internal lumen 408 of transfer sheath 400 toward aperture 506. Apparatus 100 and connector 10 may be advanced until aperture 506 rests between distal members 24 and proximal members 32 (i.e., medial portion 16) of connector 10. This configuration of first conduit 500, apparatus 100, and connector 10 is shown in FIG. 9. As described above in connection with nosecone assembly 300, nosecone balloon 302 may prevent distal members 24 from snagging on aperture 506 when distal portion 14 of connector 10 passes through the aperture. After distal members 24 have passed through aperture 506 of first conduit 500, the aperture then rests between distal members 24 and proximal members 32 of connector 10. This is because the diameters of both proximal members 32 and nosecone balloon 302 are larger than the diameter of aperture 506, thereby holding the aperture in place at medial portion 16. Preferably, the clearance between aperture 506 and medial portion 16 is minimal to ensure that proximal members 32 engage first conduit 500 evenly around the periphery of aperture 506.

FIGS. 9-13 illustrate a typical use of apparatus 100 to deliver first conduit 500 for connection to an aperture in a side wall of a second conduit, typically a patient's tubular body tissue conduit (e.g., a coronary artery requiring a bypass graft).

An aperture 602 may be made in second conduit 600 in a manner described hereinabove with respect to making aperture 506 in first conduit 500. Aperture 602 is typically made downstream from an occlusion or lesion 604 in second conduit 600. As shown in FIG. 9, nosecone balloon 302 of nosecone assembly 300 may be gradually forced into aperture 602 in a direction substantially coaxial with lumen 606 of second conduit 600. As nosecone balloon 302 passes through aperture 602, the annular recess 316 defined by the inverted proximal tapered portion 310 may shield the distal members 24 from snagging on the tissue of the second conduit 600. As long as nosecone balloon 302 remains in the introduction configuration (i.e., the folded configuration), a distal portion 318 of indicator wire 306 may extend partially beyond the proximal end portion of catheter shaft 206.

During introduction of apparatus 100 into second conduit 600, aperture 602 of the second conduit may pass over nosecone balloon 302 and settle between distal members 24 and proximal members 32 of connector 10. Aperture 602 may be disposed annularly around medial portion 16 (FIG. 1) of connector 10 next to aperture 506 of first conduit 500.

As shown in FIG. 10, the next step in the use of apparatus 100 may be to inflate nosecone balloon 302 by introducing fluid into catheter lumen 216 that flows into the nosecone balloon via nosecone shaft lumen 312. As nosecone balloon 302 expands (i.e., moves from the introduction, or folded, configuration to the removal, or unfolded, configuration), distal tip 308 moves distally into lumen 606 of second conduit 600, and the proximal tapered portion 310 returns to an unfolded condition. In the unfolded condition, distal members 24 of connector 10 are exposed within lumen 606 of second conduit 600. The distal advancement of distal tip portion 308 also advances indicator wire 306 into catheter 206. The position of indicator wire 306 with respect to catheter 206 provides a visual indication that nosecone balloon 302 has successfully moved to the removal, or unfolded, configuration.

A next step in the use of apparatus 100 is to relieve the pressure (e.g., drain the expansion fluid) from nosecone balloon 302, as shown in FIG. 11. Nosecone balloon 302 is flexible, which allows the portion of apparatus 100 that includes balloon catheter 200, connector 10, and a proximal portion of nosecone assembly 300 to be turned to a position at approximately a 90 degree angle with respect to lumen 606 of second conduit 600. As described hereinabove, connector 10 is positioned close to distal end 212 of balloon 202 to prevent dilation of second conduit 600 when balloon 202 is inflated.

A next step in the use of apparatus 100 is to inflate balloon 202 as shown in FIG. 12. Balloon 202 is inflated by introducing fluid into the balloon from catheter lumen 208. In order to create the greater deflection of the distal and proximal ends of connector 10 as described above with respect to FIGS. 3 and 4, balloon 202 has an inflated diameter larger than connector 10 to enlarge the connector. Inflation of balloon 202 causes connector 10 to annularly enlarge by enlarging cell portions 12 (FIGS. 3 and 4) in the annular direction. In addition, proximal portion 18 and distal portion 14 of connector 10 are deflected radially outward beyond medial portion 16 of the connector. These two actions, i.e., the overall annular enlargement of connector 10 and the relatively greater annular enlargement of portions 18 and 14, decrease the axial spacing between portions 18 and 14, and more particularly decrease axial spacing 36 between distal members 24 and proximal members 32 (FIG. 4). Upon inflation of balloon 202, members 32 and 24 engage the inner surfaces of conduits 500 and 600, respectively. Free ends 34 of proximal members 32 preferably penetrate the inner surface of the side wall of first conduit 500 around the periphery of aperture 506. Free ends 26 of distal members 24 preferably penetrate the inner surface of the side wall of second conduit 600 around the periphery of aperture 602. Proximal members 32 and distal members 24 therefore press apertures 506 and 602 together, creating a seal therebetween. In another example, free ends 34 may penetrate entirely through the side wall of first conduit 500, and/or free ends 26 may penetrate entirely through the side wall of second conduit 600. In yet another example, free ends 34 may engage but not penetrate the inner surface of the side wall of first conduit 500, and/or free ends 26 may engage but not penetrate the inner surface of the side wall of second conduit 600, respectively.

A next step in the use of apparatus 100 is to deflate balloon 202 and withdraw all of the elements 200 and 300 from the patient (e.g., by pulling them proximally out of first conduit 500). Subsequently, distal end portion 502 of first conduit 500 may be tied off with, for example, a ligature 510 to direct flow from first conduit 500 into second conduit 600. This leaves the side wall of first conduit 500 connected to the side wall of second conduit 600 by enlarged connector 10, as shown in FIG. 13. (It will be appreciated that FIG. 13 is greatly simplified in that it only shows the portion of the connector and the anastomosis that are in the plane of the paper on which FIG. 13 is drawn. The connector and anastomosis are in fact fully annular, all the way around the communicating apertures in the side walls of conduits 500 and 600. FIG. 4 is thus a more complete depiction of the connector in the state of the patient shown in FIG. 13.) In particular, in this example connector 10 provides a side-to-side anastomosis between first conduit 500 and second conduit 600. Body fluid from first conduit 500 is able to flow into second conduit 600 via this connection. It will be appreciated that the direction of fluid flow is entirely arbitrary, and that in another application of the invention the fluid flow direction could be opposite to that just described.

Connector 10 presses aperture 506 of first conduit 500 against aperture 602 of second conduit 600 around the peripheries of the two apertures, thereby preventing body fluid from leaking out of conduits 500 and 600. Connector 10 also prevents first conduit 500 from pulling away from the side wall of second conduit 600. It will be appreciated that the apparatus and methods described herein may also be useful for making an end-to-side anastomosis between a first conduit 500 and a second conduit 600.

It will also be appreciated that an additional anastomosis may be made between first conduit 500 and either second conduit 600 or a different body tissue conduit in the patient. For example, rather than tying off distal end portion 502 of first conduit 500 with a ligature as shown in FIG. 13, an additional anastomosis may be made between the distal end portion of the first conduit and either a location along second conduit 600 distal to the anastomosis shown in FIG. 13 or a different body tissue conduit. In one example, the additional anastomosis may be an end-to-side anastomosis between the open distal end of first conduit 500 and the side wall of the other conduit (i.e., second conduit 600 or a different body tissue conduit). In another example, the additional anastomosis may be a side-to-side anastomosis, and therefore distal end portion 502 may be tied off using a ligature.

As described hereinabove in connection with FIG. 5A, in situations in which a first conduit having a small inner diameter (e.g., about 2.0 mm or less) is to be attached to a second conduit, it may be desirable to limit the expansion of at least a portion of expansion balloon 202. For example, limiting the expansion of at least a portion of balloon 202 proximal to connector 10 may prevent the expansion balloon from dilating the first conduit. FIG. 14 illustrates a sleeve 700 that may be used for such a purpose. As shown in the FIG., sleeve 700 may be positioned annularly around a portion of expansion balloon 202 proximal to connector 10. Thus, when balloon 202 expands (as shown, for example, in FIG. 12), a portion of the expansion balloon proximal to connector 10 may be constrained by sleeve 700.

Sleeve 700 may be constructed from a rigid, high strength material having a thin wall, such as, for example, polyimide. Preferably, the wall thickness of sleeve 700 is equal to or less than about 0.003 inches. In a situation in which both the first and second conduits have small inner diameters (e.g., about 2.0 mm or less), for example, sleeve 700 may have an inner diameter of about 0.078 inches and a wall thickness of about 0.001 inches. Connector 10 may have an initial (i.e., unexpanded) inner diameter of about 0.030 inches. Proximal members 32 may have an unexpanded diameter of about 0.075 inches, distal members 24 may have an unexpanded diameter of about 0.055 inches, and nosecone balloon 302 may have a diameter of about 0.060 inches.

Sleeve 700 may be lubricous on its outer surface. In one embodiment, sleeve 700 may be constructed from a layered tube. For example, the inner layer of sleeve 700 may be constructed of a rigid, high strength material such as, for example, polyimide, to restrict the expansion of balloon 202. The outer layer of sleeve 700 may be constructed of a lubricous material such as, for example, polytetrafluoroethylene (“PTFE”).

Alternatively, grip 150 may be used to constrain balloon 202 instead of sleeve 700. For example, grip 150 may be constructed such that it is of a sufficient length to both act as a grip for apparatus 100 and a sleeve for balloon 202.

As shown in FIG. 15, sleeve 700 may be used along with another embodiment of a connector. Connector 800 may have proximal members 832 that are shorter in length than proximal members 32 of connector 10. Proximal members 832 may have a diameter that is smaller than that of nosecone balloon 302 shielding distal members 824. Such a configuration (i.e., diameter of proximal members 832 is less than diameter of nosecone balloon 302) is possible because sleeve 700 acts as a “stop” to trap first conduit 500 between distal members 824 and proximal members 832 during loading. Thus, when a sleeve 700 having a diameter larger than that of nosecone balloon 302 is positioned over connector 800, as shown in the FIG., proximal members 832 need not have a diameter larger than that of the nosecone balloon. A benefit of a connector 800 having shorter proximal members 832 is that there is a reduced possibility of the proximal members overexpanding the first conduit when the connector is expanded.

To make an anastomosis such as that shown in FIG. 13, a physician may estimate the length of a first conduit (e.g., graft conduit) necessary for creating the anastomosis. This may be difficult to measure accurately due to the difference in size between a deflated heart (e.g., during the surgical procedure) and a pressurized heart (e.g., after the surgical procedure) or due to the reorientation of some of a patient's organs during surgery to gain access to the anastomosis site. Thus, a physician may be faced with either tension on the graft if the graft is too short or a tendency for the graft to kink if the graft is too long.

FIGS. 16-19 show illustrative apparatus for reducing the tension on an anastomosis and/or for defining a kink-free path for the graft conduit to follow. (FIGS. 16-19 are greatly simplified views of the heart to facilitate the definition of the apparatus.) Such apparatus may include a U-shaped channel. The length (e.g., short or long) and shape (e.g., straight or curved) of the U-shaped channel may be varied depending on the desired result. For example, a long, curved channel 900, as shown in FIG. 18, may be positioned over top of a first conduit, such as first conduit 500. Channel 900 defines a curved path which may reduce the likelihood that first conduit 500 will kink by taking up the excess length in the first conduit. The inner surface 902 (FIG. 16) of channel 900 may include a rough surface to anchor first conduit 500 to the channel. In another embodiment, inner surface 902 of channel 900 may be coated with an adhesive to anchor first conduit 500 to the channel. As shown in cross-sectional view in FIG. 16, channel 900 may anchor first conduit 500 to the surface of heart 903 (e.g., the myocardium). Channel 900 may be constructed of any high strength material, such as, for example, stainless steel. Other illustrative materials may include, for example, tantalum, tungsten, platinum, other steels, and nitinol.

Channel 900 may be anchored to heart 903 using fine barbs 904. However, barbs 904 are merely illustrative, and channel 900 may be anchored to heart 903 using any other suitable technique for anchoring the channel. For example, as shown in FIG. 17, channel 900 may be anchored to heart 903 using adhesive pads 906.

In situations in which the physician underestimated the length of first conduit 500, a short, straight channel 1000 may be used to reduce the tension on the short graft at the anastomosis site, as shown in FIG. 19. As shown, the tension in first conduit 500 is maintained between channel 1000 and an upstream anastomosis, effectively acting as a “strain relief” or “support” for connector 10. Channel 1000 is substantially similar to channel 900. However, in addition to being short and straight, channel 1000 may also be positioned along first conduit 500 at a location close to the anastomosis site to reduce the tension on first conduit 500 at the anastomosis site. In addition, a short, straight channel such as channel 1000 may also be used to strengthen the anastomotic connection between the first and second conduits.

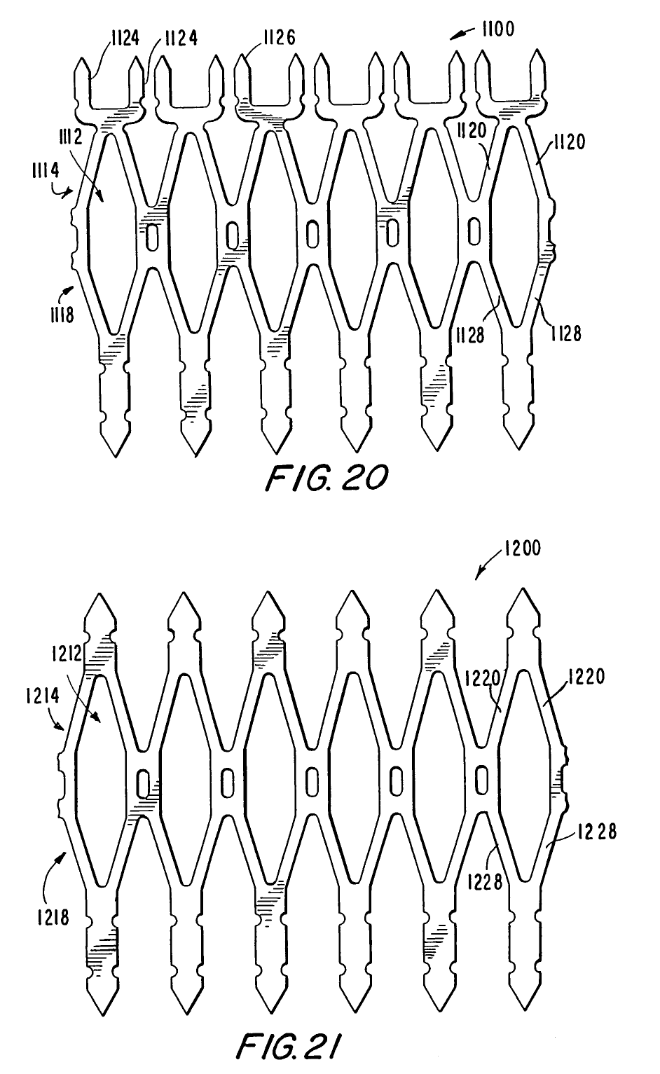

Various alternative embodiments of a connector in accordance with the invention are now described. The connectors in FIGS. 20-23 (shown in planar development) are all suitable for use with the apparatus and methods shown in FIGS. 5A-19 to provide an anastomosis between an aperture in a side wall of a graft conduit and an aperture in a side wall of a patient's body tissue conduit. The connectors in FIGS. 20-23 are of a similar size and cross-section as connector 10 (FIGS. 1-4), and the connectors are constructed of the same material or materials as connector 10. The differences between the embodiments of the connectors shown in FIGS. 20-23 and connector 10 are made apparent in the description that follows.

An illustrative embodiment of a connector 1100 in accordance with the invention is shown in FIG. 20. Connector 1100 is substantially similar to connector 10 (FIG. 1). However, a difference between the two connectors is that a cell portion 1112 of connector 1100 may have two distal members 1124, instead of only one distal member 24 as in connector 10 (FIG. 1) This aspect of connector 1100 improves the reliability of the distal members 1124 to engage the inner surface of the second conduit (e.g., a patient's body tissue conduit). For example, in a situation in which a distal member 1124 does not engage the inner surface of the second conduit after connector 1100 has been expanded, there may be a “bulge” of tissue at the location of that distal member. This bulge may result in a partial occlusion of the opening between the second conduit and the first conduit. Thus, additional distal members 1124 increase the likelihood of a distal member engaging the inner surface of the second conduit, while reducing the likelihood of a partial occlusion created by a bulge of tissue.

Another difference between connector 1100 and connector 10 is that cell portions 1112 have a slightly different shape than cell portions 12 of connector 10. For example, members 1120 may be connected to one another at distal end portion 1114, and members 1128 may be connected to one another at proximal end portion 1118.

An illustrative embodiment of a connector 1200 in accordance with the invention is shown in FIG. 21. Connector 1200 is substantially similar to connector 10 (FIG. 1). However, as described above in connection with connector 1100, a difference between the two connectors is that cell portions 1212 of connector 1200 have a slightly different shape than cell portions 12 of connector 10. For example, members 1220 may be connected to one another at distal end portion 1214, and members 1228 may be connected to one another at proximal end portion 1218.

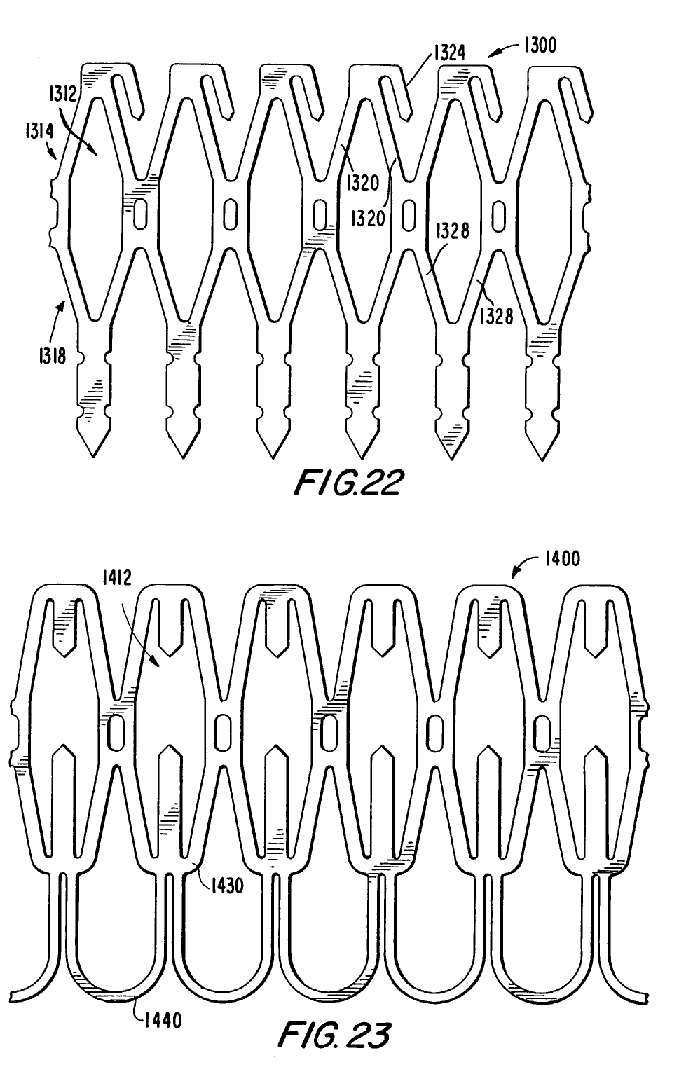

An illustrative embodiment of a connector 1300 in accordance with the invention is shown in FIG. 22. Connector 1300 is substantially similar to connector 10 (FIG. 1). However, as described above in connection with connectors 1100 and 1200, a difference between the two connectors is that cell portions 1312 of connector 1300 have a slightly different shape than cell portions 12 of connector 10. For example, members 1320 may be connected to one another at distal end portion 1314, and members 1328 may be connected to one another at proximal end portion 1318.

Another difference between connector 1300 and connector 10 is that distal members 1324 may be external to cell portion 1312, instead of within cell portion 12 as with connector 10. An advantage to such a configuration of distal members 1324 is that it may be easier to form connector 1300 into the desired shape when the connector is placed around a mandrel. For example, it may be easier to pry distal members 1324 outward into the desired formation, since the members lie outside cell portions 1312.

An illustrative embodiment of a connector 1400 in accordance with the invention is shown in FIG. 23. Connector 1400 is substantially similar to connector 10 (FIG. 1). However, connector 1400 may include U-shaped members 1440. One end of a member 1440 may be attached to annularly extending member 1430 of a cell portion 1412, and the other end of U-shaped member 1440 may be attached to another member 1430 of an annularly adjacent cell portion 1412. Members 1440 may improve the rigidity of connector 1400. For example, members 1440 may prevent a cell portion 1412 from twisting with respect to the two annularly adjacent cell portions 1412.

It will be understood that the foregoing is only illustrative of the principles of the invention, and that still other modifications can be made by those skilled in the art without departing from the scope and spirit of the invention. For example, the various materials and dimensions mentioned herein are only examples, and other materials and dimensions can be used, if desired.

The invention claimed is:

1. A method of producing a hollow annular anastomotic connection between a first aperture in a side wall of a graft conduit and a second aperture in a side wall of a body tissue conduit in a patient using a hollow annular connector having a first axial portion, a second axial portion, and a medial portion therebetween, wherein the first axial portion has a plurality of first members and the second axial portion has a plurality of second members, wherein the diameters defined by both the first members and second members are larger than the diameter of the medial portion, and wherein an axial spacing is defined between the first members and the second members, comprising:
introducing the hollow annular connector into the graft conduit so that the first axial portion of the connector is disposed inside the graft conduit and the second axial portion of the connector extends out of the graft conduit via the first aperture;
approximating the first and second apertures so that the second axial portion of the connector extends into the body tissue conduit via the second aperture; and
deforming the connector so that the axial spacing decreases and so that the first and second members press together the side walls of the body tissue conduit and the graft conduit annularly around the first and second apertures.

2. The method defined in claim 1 wherein the graft conduit has first and second segments that extend in respective opposite directions along the graft conduit from the first aperture, wherein body fluid will flow in the first segment after the connection has been made, wherein the second segment has a third aperture spaced from the first aperture, and wherein the introducing comprises:
inserting the connector into the second segment via the third aperture and passing the connector along the inside of the second segment to the first aperture.

3. The method defined in claim 2 further comprising:
before the inserting, providing an introduction structure having a tapered portion configured to shield the second axial portion of the connector, and
wherein the inserting the connector comprises inserting the tapered portion of the introduction structure into the second segment via the third aperture while shielding the second axial portion of the connector.

4. The method defined in claim 2 further comprising:
before the inserting, providing a selectively inflatable balloon that is disposed annularly within the connector, and
wherein the inserting the connector comprises inserting the balloon that is disposed annularly within the connector into the second segment via the third aperture.

5. The method defined in claim 2 further comprising closing the second segment after the connection has been made.

6. The method defined in claim 1 further comprising:
shielding the second axial portion of the connector during at least part of the introducing.

7. The method defined in claim 6 further comprising:
unshielding the second axial portion of the connector after the approximating and before the deforming.

8. The method defined in claim 1 wherein the deforming comprises:
annularly enlarging the connector.

9. The method defined in claim 1 wherein the deforming comprises:
axially shortening the connector.

10. The method defined in claim 1 wherein the connector is disposed annularly around a selectively inflatable balloon, and wherein the deforming comprises:
inflating the balloon.

11. The method defined in claim 10 further comprising:
after the deforming, deflating the balloon.

12. The method defined in claim 11 further comprising:
after the deflating, removing the balloon from inside the connector and from the patient.

13. The method defined in claim 1 wherein the plurality of first members have free end portions configured to engage the side wall of the graft conduit, and wherein the plurality of second members have free end portions configured to engage the body tissue conduit, and wherein the deforming comprises:
engaging the side wall of the graft conduit with the free end portions of the first members and engaging the side wall of the body tissue conduit with the free end portions of the second members.

14. Apparatus for inserting a hollow annular connector, around which a first aperture in a side wall of a graft conduit is disposed, into a second aperture in a side wall of a patient's body tissue conduit from outside the body tissue conduit comprising:
a tapered portion configured to shield a portion of the connector during insertion wherein the tapered portion is constructed of a flexible material; and
a shaft portion extending from the tapered portion wherein the shaft portion is constructed of the flexible material.

15. The apparatus defined in claim 14 wherein the tapered portion defines an annular recess for shielding the portion of the connector during insertion.

16. The apparatus defined in claim 15 wherein the shaft portion extends from the annular recess.

17. The apparatus defined in claim 14 further comprising:
a tubular structure having a lumen that is in fluid communication with an internal cavity of the tapered portion and a lumen of the shaft portion.

18. The apparatus defined in claim 17 wherein a portion of the shaft portion having an open end is disposed annularly within the tubular structure, and wherein the open end is attached to the tubular structure.

19. Apparatus for producing a hollow annular anastomotic connection between a first aperture in a side wall of a graft conduit and a second aperture in a side wall of a body tissue conduit in a patient comprising:
a hollow annular connector having a first axial portion configured to engage the graft conduit and a second axial portion configured to engage the body tissue conduit, the connector being deformable from a first configuration to a second configuration having an enlarged annular dimension;
a balloon structure having a selectively inflatable balloon configured for positioning within the connector to deform the connector from the first configuration to the second configuration;
an introduction structure having a first configuration and a second configuration, the first configuration having a tapered portion defining an annular recess for shielding the second axial portion of the connector, and the second configuration having a smaller dimension, wherein the introduction structure moves to the second configuration to unshield the second axial portion of the connector; and
a rigid tubular structure disposed annularly around the balloon adjacent the first axial portion of the connector configured to restrict the inflation of the balloon.

20. Apparatus for producing a hollow annular anastomotic connection between a first aperture in a side wall of a graft conduit and a second aperture in a side wall of a body tissue conduit in a patient comprising:
an introduction structure wherein the introduction structure has a tapered portion and a shaft portion extending from the tapered portion, and wherein the tapered portion has a diameter that is greater than the diameter of the first aperture; and
a hollow annular connector wherein the connector has a plurality of first members configured to engage the graft conduit and a plurality of second members that are shielded by the tapered portion during insertion and that are configured to engage the body tissue conduit, and wherein the first members have a diameter that is greater than the diameter of the tapered portion.

21. An apparatus for anchoring a graft conduit to a tissue surface in a patient, after an anastomotic connection has been made between the graft conduit and a body tissue conduit in the patient, comprising a channel that is configured to receive the graft conduit within the confines of the channel, and wherein the channel has anchoring structures to engage the tissue surface.

22. The apparatus defined in claim 21 wherein the anchoring structures are barbs.

23. The apparatus defined in claim 21 wherein the anchoring structures are adhesive pads.

24. The apparatus defined in claim 21 wherein the channel is curved so that the channel takes up excess length of the graft conduit.

25. The apparatus defined in claim 21 wherein the channel is straight.

26. The apparatus defined in claim 25 wherein the channel is positioned adjacent to the anastomotic connection to reduce the tension on the graft conduit at the anastomotic connection.

27. The apparatus defined in claim 21 wherein an inner surface of the channel is rough so that the inner surface engages the graft conduit.

28. The apparatus defined in claim 21 wherein an inner surface of the channel is coated with an adhesive so that the inner surface engages the graft conduit.

29. The apparatus defined in claim 21 wherein the channel is U-shaped.
